(19) United States
(12) Patent Application Publication
(10) Pub. No.: US 2025/0078989 A1
(43) Pub. Date: Mar. 6, 2025

(54) SYSTEMS AND METHODS FOR AUTOMATIC INSURANCE VERIFICATION (AIV)

(71) Applicant: CCS Medical, Inc., Clearwater, FL (US)

(72) Inventors: Ellen Eames, Clearwater, FL (US); Teresa Brammer, Clearwater, FL (US); Ramzi Khoury, Clearwater, FL (US); Dan Moran, Clearwater, FL (US)

(21) Appl. No.: 18/240,912

(22) Filed: Aug. 31, 2023

(51) Int. Cl. G16H 40/20; G06Q 40/08; G16H 10/60

(52) U.S. Cl. CPC G16H 40/20; G06Q 40/08; G16H 10/60

(57) ABSTRACT

The present disclosure generally relates to systems and methods for automatically verifying the medical benefits information of a patient. The present systems and methods automatically verify a patient's medical insurance information by creating a patient account data structure with predetermined data fields. The data fields are then populated with information from a memory device that contains data structures with medical benefits information arranged by an insurance provider identifier.

### BACKGROUND

For a medical service provider to receive compensation for services provided from a medical insurance payor, the medical service provider must determine whether a patient is eligible for medical insurance coverage. The medical service provider also has to verify the medical benefits for which the medical insurance payor will provide compensation. To this end, medical service providers request health insurance information from patients during registration to identify the patient's medical insurance payor and the patient's insurance policy with the payor.

Currently, once the medical service provider receives the information via a patient intake form, for example, the medical service provider must then engage in a series of manual, iterative processes to confirm the medical insurance eligibility of the patient and verify the medical benefits for which the patient is entitled. These manual processes not only consume a significant amount of time, but they also drastically increase the likelihood of human error, which delays the medical insurance provider from being compensated by the medical insurance payor for services the medical services provider has already rendered to the patient.

Additionally complicating the process of verifying the medical benefits for which the patient is entitled is the fact that medical benefits information often exists in unstructured data formats. Furthermore, each participant in the medical insurance reimbursement process (e.g., providers, medical claim clearinghouses, and public and private payors) may use differently formatted data structures. Thus, although the process of medical service reimbursement often occurs over a computer network, the challenging data environment has limited the ability to automate the process of determining a patient's medical insurance eligibility and verifying the patient's medical benefits information.

### SUMMARY

The present disclosure generally relates to systems and methods for automatically determining a patient's insurance eligibility and verifying the medical benefits information of a patient. The example systems and methods are configured to automatically determine the eligibility and medical benefits information of a patient who possesses medical insurance. The example systems and methods are configured to automatically verify a patient's medical insurance information using one or more Application Programming Interfaces (“APIs”) that integrates with other APIs within a medical insurance payment network.

The example systems and methods also provide for the automated order and reorder of certain medical equipment. Specifically, the systems and methods, upon verifying a patient's medical insurance, are configured to determine whether the patient is eligible for certain medical equipment or referrals based on the patient's medical insurance. For example, different medical insurance policies have pre-determined times at which a patient can replace certain medical equipment. The example systems and methods are configured to determine whether a patient is eligible for reimbursement under his or her medical insurance policy based on when he or she last purchased certain medical equipment. In some instances, the systems and methods may prevent a patient from replacing or reordering medical equipment when the time since the patient last received the medical equipment is below a reimbursement threshold. The automated rejection of the medical equipment reorder may prompt automated or manual follow ups as to the reason for the reorder, which can be approved under certain circumstances, such as to replace malfunctioning equipment.

The example systems and methods also provide for the collection and preservation of medical data. For example, the systems and methods automate determining whether a patient has received the same or similar piece of equipment previously. One outcome of this collection and preservation of medical data is that it could provide important medical history to a provider.

The example systems and methods are also configured to automatically verify the medical benefits information of a patient by comparing the patient's insurance information with information stored in a memory device having multiple data structures that contain medical benefits data associated with an insurance provider. Thus, once the example systems and methods receive patient information from a provider, the example systems and methods create a patient account data structure based on a data structure template and then populate the medical benefits data fields with data from the memory device. The patient account data structure is then stored in a patient information database for later use by an entity.

In light of the disclosure set forth herein, and without limiting the disclosure in any way, in an aspect of the present disclosure, which may be combined with any other aspect, or portion thereof, described herein a system for automatically verifying medical benefits information includes a memory device having a plurality of data structures, where the plurality of data structures include predetermined data fields, which comprise at least medical benefit data fields and an insurance provider identifier data field. The system also includes a server communicatively coupled to the memory device via a network. The server is configured to receive a set of patient information associated with the patient from a healthcare provider computer over the network. The set of patient information includes at least an insurance provider identifier. The server creates a patient account data structure using a data structure template by recording or writing the insurance provider identifier received in the set of patient information into the insurance provider identifier data field, where the data structure template has the same data fields as the plurality of data structures in the memory device. The server also queries the memory device for data structures with the same insurance provider identifier as the insurance provider identifier of the patient account and after locating a data structure in the memory device with the same insurance provider identifier, the server automatically records or writes the data in the medical benefits data fields of the data structure in the memory device into the medical benefits data fields of the patient account data structure. The server then stores the patient account data structure in a patient information database that is communicatively coupled via the network to the server.

In a second aspect of the present disclosure, which may be combined with any other aspect, or portion thereof, described herein, a set of patient information further comprises a first name, a last name, a date of birth, a member identification number, or a group identification number associated with the patient.

In a third aspect of the present disclosure, which may be combined with any other aspect, or portion thereof, described herein, the predetermined data fields of a plurality of data structures in the memory device and a data structure template comprise: an insurance status; an insurance type; an insurance policy type; an insurance provider company name; a set of insurance company contact information; an insurance policy number; an insurance category number; an insurance group number; an insurance term date; a set of policyholder information; co-pay data; reimbursement data; deductible data; out-of-pocket data; a set of medical equipment guidelines; a set of prescription authorizations.

In a fourth aspect of the present disclosure, which may be combined with any other aspect, or portion thereof, described herein, when populating medical benefit data fields of a patient account, the server is further configured to, upon an unsuccessful match, automatically transmits a message to the healthcare provider computer, via the network. The message prompts a healthcare provider to request medical benefit data from the patient.

In a fifth aspect of the present disclosure, which may be combined with any other aspect, or portion thereof, described herein, an insurance provider identifier is an insurance group identification number.

In a sixth aspect of the present disclosure, which may be combined with any other aspect, or portion thereof, described herein, the data structures in the memory device are organized by insurance group identification number.

In a seventh aspect of the present disclosure, which may be combined with any other aspect, or portion thereof, described herein, a method for automatically determining medical benefits information includes receiving a set of patient information associated with a patient from a healthcare provider computer over a network. The set of patient information includes at least an insurance provider identifier. The method also includes creating a patient account data structure using a data structure template by recording the insurance provider identifier received in the set of patient information into an insurance provider identifier data field, where the data structure template includes predetermined data fields. In an embodiment, the predetermined data fields comprise at least medical benefit data fields and an insurance provider identifier data field. In addition to above, the method includes querying the memory device for data structures with the same insurance provider identifier as the insurance provider identifier of the patient account and after locating a data structure in a memory device with the same insurance provider identifier, automatically recording the data in the medical benefits data fields of the data structure in the memory device into the medical benefits data fields of the patient account data structure. The method further includes storing the patient account data structure in a patient information database that is communicatively coupled to the server.

In another aspect, any of the features, functionality and alternatives described in connection with any one or more of FIGS. 1 to 8 may be combined with any of the features, functionality, and alternatives described in connection with any other of FIGS. 1 to 8.

In light of the present disclosure and the above aspects, it is therefore an advantage of the present disclosure to automatically determine the eligibility of a patient for medical insurance coverage.

It is another advantage of the present disclosure to create a patient account data structure for automatically verifying a patient's medical benefits information.

It is a further advantage of the present disclosure to update patient accounts with the patient's medical benefits information and then store the updated patient account in a memory device.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 illustrates a diagram of a medical insurance eligibility and benefit verification system, according to an example embodiment of the present disclosure.

FIG. 2 illustrates a method for automatically determining an eligibility of a patient's insurance and verifying medical benefits information, according to an example embodiment of the present disclosure.

FIG. 3 illustrates a method for determining an eligibility of a patient's insurance, according to an example embodiment of the present disclosure.

FIG. 4 illustrates a method of automatically recording medical benefits information to a patient account data structure, according to an example embodiment of the present disclosure.

FIG. 5 illustrates an example of data structures disclosed herein, according to an example embodiment of the present disclosure.

FIG. 6 illustrates APIs of the medical insurance eligibility and benefit system, according to an example embodiment of the present disclosure.

FIG. 7 illustrate an example of a server configured to create and update a patient account data structure with medical benefits information from data structures stored in a memory device and then store the updated patient account data structure in a patient information storage device, according to an example embodiment of the present disclosure.

FIG. 8 illustrates an example data structure template, according to an example embodiment of the present disclosure.

### DETAILED DESCRIPTION

The present disclosure generally relates to systems and methods for automatically verifying medical benefits insurance information and determining a patient's insurance eligibility. The example systems and methods are configured to use one or more Application Programming Interfaces (“APIs”) that are configured to receive patient medical insurance information from medical referral applications. The APIs are configured to query one or more insurance data structures to determining matching patient insurance information. When there is a match, the one or more APIs return an indication of the patient's medical insurance. The APIs may also return information associate with the medical insurance, such as benefits, copay amounts, restrictions, etc. In some instances, the APIs may integrate with other APIs within a medical insurance payment network

As used herein, the terms “individual”, “patient”, “client”, “user” or the like, or their plural forms, refer to any person, individual, patient, and/or client who uses the disclosed systems and methods and/or who seeks and/or who receives healthcare services, healthcare-related services, healthcare-related information, and/or any of the other services and/or products provided by the present invention.

As used herein, the terms “doctor”, “healthcare provider”, “provider”, “therapist”, “healthcare information specialist”, etc., or their plural forms, refers to any medical doctor, including any and all of the various medical specialists and/or specialties, including, but not limited to internists, orthopedists, opthamalogists, cardiologists, hematologists, endocrinologists, oncologists, ears, nose and throat specialists, neurologists, urologists, gastrointerologists, dermatologists, pediatricians), medical specialist, surgeon, surgical specialists, including any and/or forms and/or types of surgeons), physician, dentist, psychiatrist, psychologist, optometrist, podiatrist, osteopath, chiropractor, pharmacist, therapist, physical therapist, respiratory therapist, nurse, healthcare aid, nutritionist, and/or any other person, individual and/or professional who can provide healthcare, healthcare-relate, wellness and/or wellness-related services and/or products.

As used herein, the terms “insurer”, “payer”, “insurance provider”, “health insurance provider”, “life insurance provider”, “disability insurance provider”, etc., or their plural forms, refers to any insurance companies, healthcare insurance companies, disability insurance companies, property or casualty insurance companies, health maintenance organizations, healthcare providers, and any other payer and/or provider of healthcare services and/or products, who which provide and/or pay for healthcare and/or healthcare-related benefits, services, and/or products, and/or who or which provide respective health insurance, life insurance and/or disability insurance benefits, services and/or products.

Reference is also made herein to patient information. As discussed in more detail below, the patient information includes information that is given to a healthcare provider by the patient. The patient information is self-reported by a patient and includes the patient's first name, last name, date of birth, insurance identification number, and/or insurance group number. The patient information may be entered into a computer of the healthcare provider. In some instances, a patient enters their own information into the healthcare provider computer directly or remotely using a patient computer.

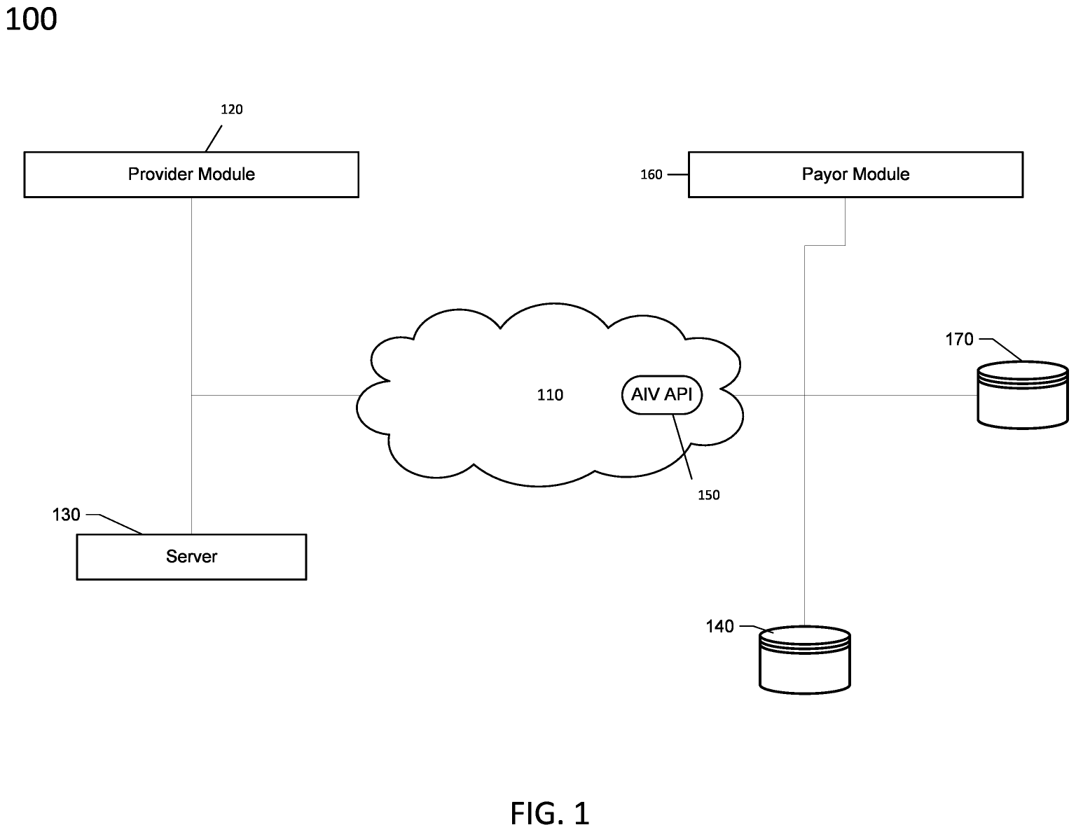

FIG. 1 illustrates an example medical insurance eligibility and benefit system 100, according to an example embodiment of the present disclosure. The system 100 includes a server 130 that is configured to automatically verify the medical insurance benefits information of a patient. The server 130 may include a single device or may be provisioned within a cloud computing or distributed computing environment. Further, while FIG. 1 shows a single server 130, the example system 100 may include multiple servers 130.

The server 130 is configured to receive a set of patient information from a provider module 120 (e.g., a healthcare provider computer) via a network 110. The provider module 120 comprises a desktop computer, a laptop computer, a smartphone, a processor, a logic controller, a computer network, or any combination thereof that is operated by a healthcare provider. In one embodiment, the provider module 120 includes software defined by machine readable instructions. Execution of the instructions by a processor of the provider module 120 provides a prompt and/or data fields for a user to input information related to a patient. The information may include the patient's first name, last name, date of birth, insurance identification number, insurance group information, etc.

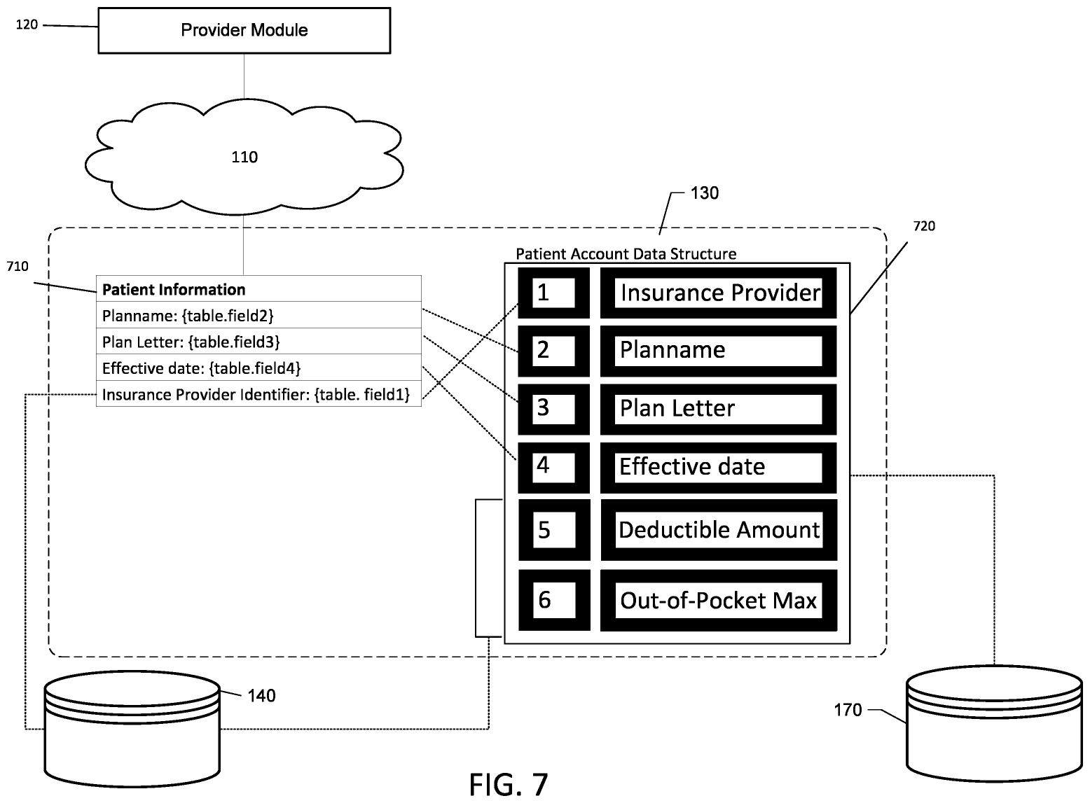

In one embodiment, after receiving a call from an AIV API 150, the provider module 120 communicates a standardized set of the received patient information to the server 130. The server 130 then creates a patient account data structure (e.g., the patient account data structure 720 of FIG. 7) based on a data structure template. In another embodiment, the provider module 120 directly interfaces with the server 130, thereby enabling the server 130 to directly request patient information from the provider module 120.

As shown in FIG. 1, the server 130 may be communicatively coupled to a memory device 140 via the network 110. Alternatively, the server 130 may be directly coupled to the memory device 140. The memory device 140 is configured to store data structures that contain information in predetermined data fields. The server 130 is configured to query the memory device 140 to identify data structures that contain, for example, information with the same insurance provider identifier as the patient account data structure.

In an embodiment, after updating a patient account data structure, the server 130 is configured to store the updated patient account data structure in a patient information database 170. The example patient information database 170 is communicatively coupled to other components in the medical insurance eligibility and benefit system 100 for use by the other components. In another embodiment, the server 130 transmits the updated patient account data structure to the provider module 120 directly or via the network 110.

FIG. 1 also shows that the system 100 includes a payor module 160. In an example embodiment, a payor module 160 enables for the automated reordering of certain medical equipment (or medical services). Upon determining an account is valid, the AIV API 150 utilizes the medical benefits information associated with a patient account data structure to determine whether a patient is authorized to receive certain medical equipment. When authorized, the AIV API 150 is configured to transmit an authorization message to the payor module 160, which prompts a payor to pay the healthcare provider for the authorized medical equipment.

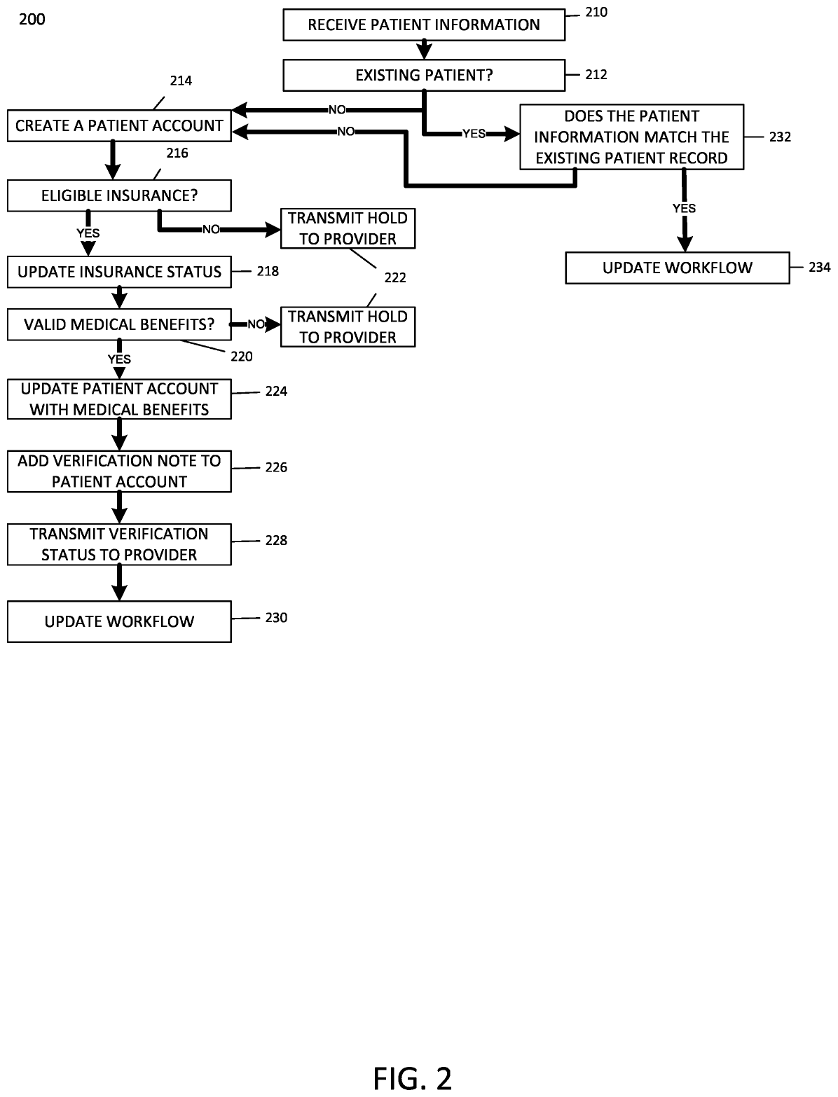

FIG. 2 shows a method of automatically determining the eligibility of a patient's medical insurance information and verifying the medical benefits information of a patient, according to an example embodiment of the present disclosure. In some instances, the AIV API 150 includes software or other predefined algorithms specified by machine readable instructions that, when executed, performs the operations disclosed herein. In an embodiment, the AIV API 150 receives patient information (block 210). The AIV API 150 then determines whether the received patient information corresponds to patient information that already exists in the patient information database 170 (block 212). When the patient information already exists in the patient information database 170, the AIV API 150 compares the received patient information to the information that exists in the patient information database 170 to determine whether the information matches (block 232). When there is an exact match, the workflow for determining eligibility and verifying the medical benefits information of the patient is updated (block 234). However, when the patient information is not contained in the patient information database 170, the AIV API 150 creates a patient account data structure and populates the patient account data structure with the received patient information (block 214).

The AIV API 150 next determines whether the information in the patient account data structure corresponds to eligible insurance coverage provided by a payor (block 216). When the information does not correspond to eligible insurance, the AIV API 150 transmits a hold message to the provider module 120, which informs the healthcare provider that the patient does not possess eligible medical insurance (block 222). When the information does correspond to eligible insurance, the AIV API 150 updates the insurance status as eligible (block 218).

In an embodiment, the AIV API 150 then communicates with the server 130 to determine whether the patient possesses valid medical benefits based on the patient's medical insurance (block 220). When the patient does not possess valid medical benefits, the server 130 prompts the AIV API 150 to transmit a hold message to the provider module 120, which informs the healthcare provider that the patient does not possess the necessary medical benefits (block 222). When the patient does possess valid medical benefits, the server 130 updates the patient account data structure with the patient's medical benefits information (block 224).

In an embodiment, the AIV API 150 then adds a verification note to the patient account data structure (block 226). This verification notes enables a human to understand the last time a patient's medical insurance eligibility medical benefits information was last determined. The verification note may include, for example, a current date/time of the most recent verification. The verification status is then transmitted to the healthcare provider via the provider module 120 (block 228). The workflow is next accordingly updated (block 230). Updating the workflow may include transmitting a message from the server 130 to the provider module 120 indicative of the patient's medical insurance and corresponding benefits, as described in more detail below. The example method then ends or returns to block 210 when new patient information is received.

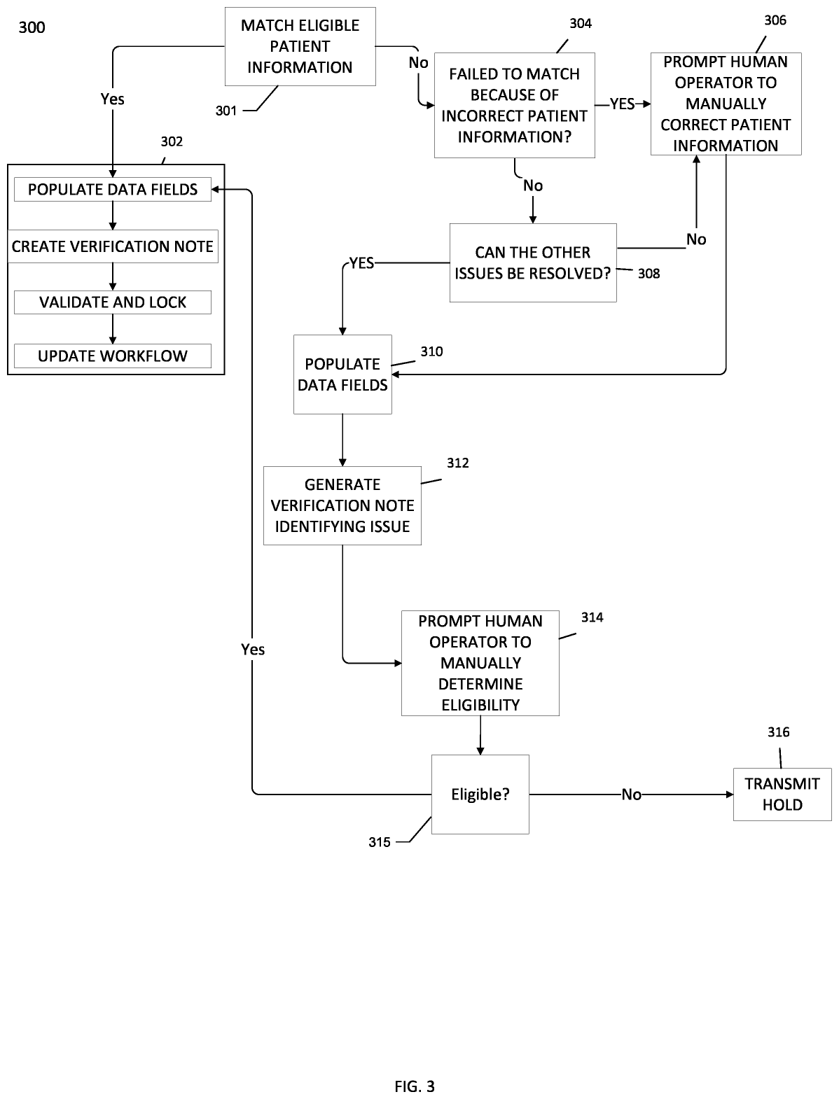

FIG. 3 illustrates an embodiment of a method used by the AIV API 150 to determine the eligibility of a patient's medical insurance coverage 300, according to an example embodiment of the present disclosure. In an embodiment, the AIV API 150 determines whether patient information matches eligible patient information (block 301). When there is a match, the AIV API 150 uses a method in a similar fashion as the method outlined in FIG. 2, which includes populating data fields of the patient account data structure, creating a verification note, validating and locking the patient account data structure, and then updating the workflow (block 302).

In an example embodiment, when there is no match, the AIV API 150 then proceeds through a series of steps to determine whether there was an error because of incorrect patient information (block 304). Errors include typographical errors, mixing information from different patients (e.g., using the first name of one patient with the last name of another), and other similar recording or manual input errors. When the failure to match is due to a typographical error, the AIV API 150 transmits a message to a human operator at the provider module 120. The message may indicate an error with the input information and include a prompt to correct the patient information (blocks 306 and 310). Additionally, the fact that an error was generated is recorded, for example, in the verification note (block 312).

When the error is not due to a typographical error, the AIV API 150 next determines if other issues related to the patient eligibility information can be automatically resolved (block 308). When the issues cannot be automatically resolved, the AIV API 150 transmits a message to a human operator at the provider module 120. The message may indicate an error with the eligibility information and include a prompt to correct the information (blocks 306 and 310). However, when the issues can be automatically resolved, the AIV API 150 populates the data fields of the patient account data structure with the information provided and then generates a verification note identifying the issue (blocks 310 and 312). This verification note prompts a human at the provider module 120 to address the remaining issues and manually determine eligibility (block 314). When the human operator determines the patient possesses eligible medical insurance (block 315), the AIV API 150 then uses a method in a similar fashion as the method outlined in FIG. 2, which includes populating data fields of the patient account data structure, creating a verification note, validating and locking the patient account data structure, and then updating the workflow accordingly (block 302). When the human operator determines the patient possesses ineligible medical insurance, the AIV API 150 transmits a hold message (block 316). The example method then ends or returns to the beginning when new patient information is received for comparison.

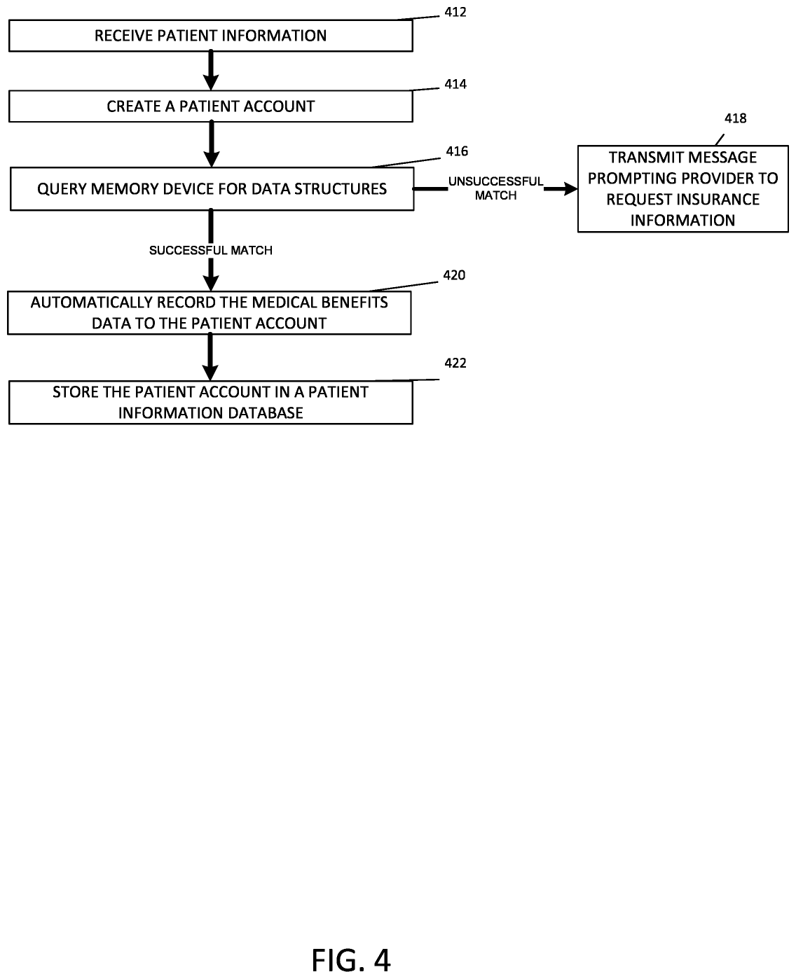

FIG. 4 illustrates a method of automatically determining the medical benefits information of a patient using data structures and a data structure template, according to an example embodiment of the present disclosure. The server 130 receives patient information from the AIV API 150 (block 412) and creates a patient account data structure based on a data structure template (block 414). Using the insurance provider identifier, the server 130 then performs a query of the memory device 140 to identify any data structures with the same insurance provider identifier (block 416). When there is a successful match, the sever 130 then automatically records the medical benefits information contained within the data structure located in the memory device into the corresponding data fields of the patient account data structure (block 420). The server 130 may also store the patient account data structure in the patient information database 170 (block 422). When there is an unsuccessful match, the server 130 prompts the AIV API 150 to transmit a message to a human operator at the provider module 120 requesting the human operator to manually input the medical benefits information into the patient account data structure (block 418). In an embodiment, the server 130 is also configured to store the patient account data structure from manual entry in the patient information database 170.

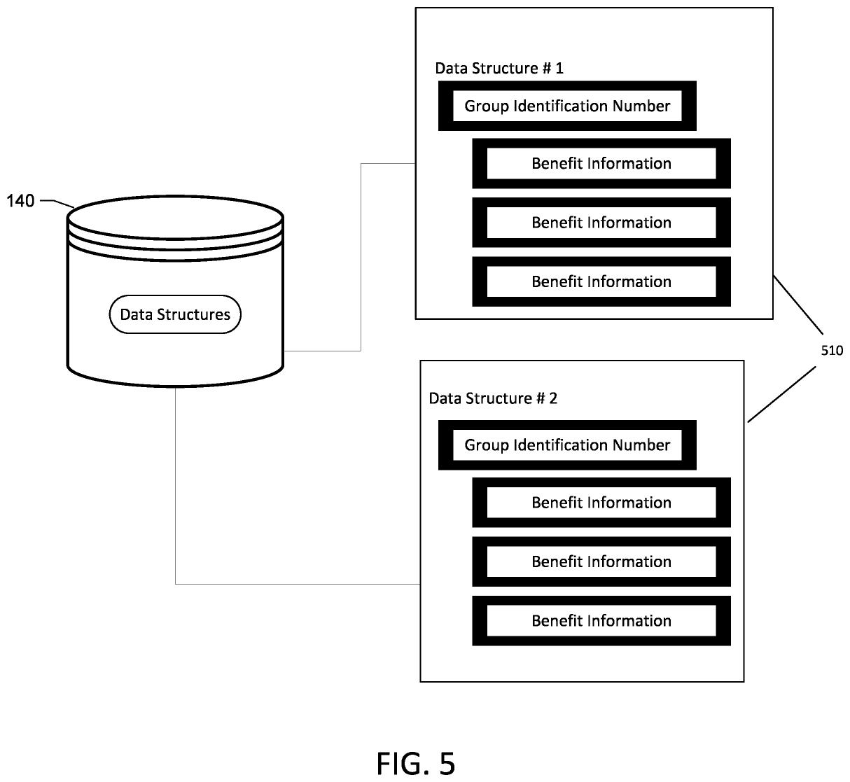

FIG. 5 illustrates information stored in the memory device 140 of FIG. 1, according to an example embodiment of the present disclosure. In an embodiment, data structures 510 are arranged in the memory device 140 by a group identification number that identifies an insurance provider. Associated with each group identification number is medical benefits information (e.g., copay amount, out-of-pocket maximum, and other similar payment information).

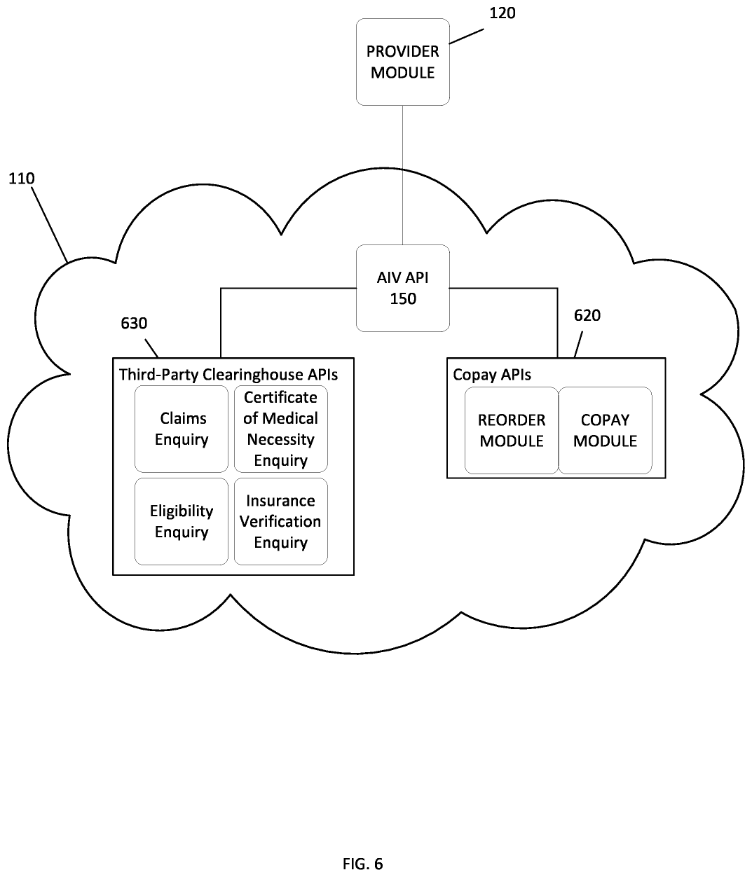

FIG. 6 illustrates a series of API communicatively coupled together in the system 100 of FIG. 1, according to an example embodiment of the present disclosure. In an embodiment, the AIV API 150 is in communication with third-party clearinghouse APIs 630. In the medical insurance eligibility and benefit system 100, clearinghouses serve as intermediaries between healthcare providers and payors and help facilitate the reimbursement of healthcare providers by payors. The APIs 630 can be communicatively coupled to third-party systems related to medical claims, certificates of medical necessity, patient eligibility, and/or insurance verification. Certain entities use software and hardware that assist in determining the eligibility of medical insurance (e.g., pVerify Claims, pVerify CMN, Eligiblity Enquiry v3, and Eligibility Enquiry Pokitdoc). Each of these entities have specific, different formats for data structures that may not be compatible among each other. Therefore, the AIV API 150 is configured to format message in the medical insurance eligibility and benefit system 100 that provides for the automation of eligibility determination regardless of which party is serving as the healthcare provider, clearinghouse, or payor. In an embodiment, the AIV API 150 is communicatively coupled to a series of Copay APIs 620. These APIs are associated with payors and help facilitate the payment of medical services rendered by the healthcare provider.

In an example, the AIV API 150 receives patient information. The AIV API 150 is configured to convert the patient information into one or more formats based on which third-party clearinghouse APIs 630 are to be accessed. The AIV API 150 transmits the formatted patient information to the corresponding third-party clearinghouse APIs 630 to determine, for example, insurance eligibility and corresponding benefits, as discussed in conjunction with FIG. 2. The server 130 uses the results from the AIV API 150 to create a patient account so that the third-party clearinghouse APIs 630 do not need to be accessed frequently within a specified time period for the same patient. Instead, the server 130 uses the patient account to provide patient eligibility and benefits information. However, after the threshold, the AIV API 150 may access the third-party clearinghouse APIs 630 to re-confirm patient insurance eligibility and/or benefits. The threshold may be two weeks, a month, two months, six months, a year, etc.

Alternatively, the third-party clearinghouse APIs 630 may transmit a message to the AIV API 150 when a patient's eligibility, insurance provider, and/or benefits change. The message may be indicative of the change in information. In some instances, the message may include the changed insurance information, which is stored by the AIV API 150 to the associated patient account data structure in the patient information database 170. In other instances, after receiving a message indicative of a change, the AIV API 150 performs the operations described in FIG. 2 to update the patient account data structure in the patient information database 170.

FIG. 7 illustrates the creation of a patient account data structure 720 based on patient information 710 received from a healthcare provider computer or module 120 via the network 110, according to an example embodiment of the present disclosure. In an embodiment, the provider module 120 transmits patient information 710 via the network 110 to the server 130, which is configured to create a patient account data structure 720 based on a data structure template. In this example, the “Planname,” “Plan Letter,” “Effective date,” and “Insurance Provider Identifier” are recorded into the corresponding data fields of the patient account data structure 720 (data fields 2, 3, 4, 1 respectively).

The server 130 is also configured to query the memory device 140 using the insurance provider identifier to identify data structures in the memory device 140 that contain the same insurance provider identifier. Upon location of matching data structures, any medical benefits information contained in the matching data structures is recorded into the corresponding data fields of the patient account data structure 720. In this example, the deductible amount and out-of-pocket maximum is recorded into the corresponding data fields of the patient account data structure 720 (data fields 5 and 6 respectively).

In one embodiment, the server 130 stores the patient account data structure 720 in the patient information database 170. Additionally or alternatively, the server 130 transmits the patient account data structure 720 to the provider module 120. Otherwise, the server 130 makes the patient account data structure 720 available to the provider module 120. The server 130 may further use the patient account data structure 720 for requests with patient information that is received from other provider modules 120.

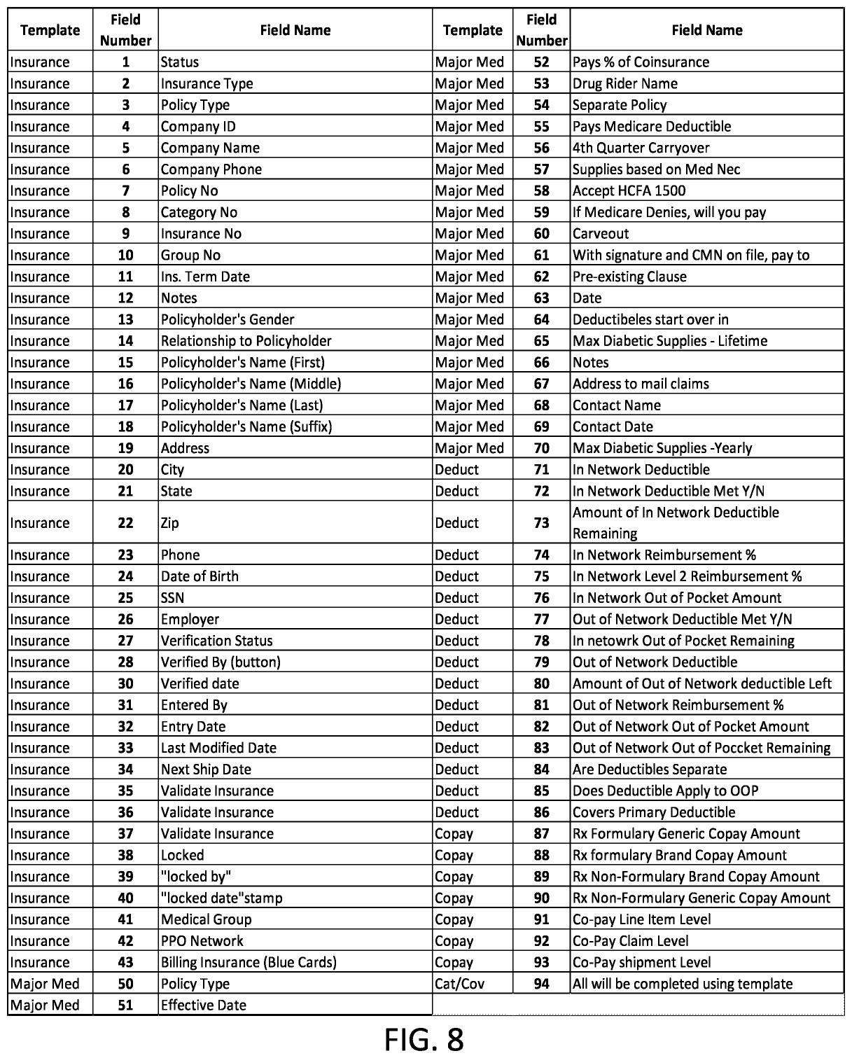

FIG. 8 is a diagram of an example data structure template 800, according to an example embodiment of the present disclosure. In an embodiment, the data structure template 800 includes data fields in a predetermined order. The predetermined order of the data fields enables for the automation of the verification of medical benefits information using the APIs and the server 130 discussed above. The data structure template 800 includes fields for insurance status, insurance type, policy type, company identifier, company name, company phone number, policy number, category number, insurance number, group number, insurance term date, and/or notes. The data structure template 800 also includes fields for the patient including gender, relationship to a policyholder, name, address, phone number, birth date, social security number, and employer. The data structure template 800 further includes fields indicative of insurance verification including verification status, verifier, verification date, and an insurance verification modification date. The data structure template 800 also includes fields for copay information, deductible information, and major medical information. It should be appreciated that the fields shown within the data structure template 800 are only exemplary and other data structure templates 800 may include additional or fewer fields.

As discussed above, the data structure template 800 may additionally include fields regarding medical equipment provided to a patient. The fields may identify a type/name of the equipment, a model number, a serial number, a distributor, etc. The fields may also specify a date the medical equipment was provided to a patient and/or an end/expiration date or a renewal date. When the server 130 receives a request for new medical equipment, the server 130 populates the corresponding fields after receiving an indication that the medical equipment is approved. In some instances, the server 130 may determine the approval using other populated fields of the data structure template 800. Later, when a request for replacement equipment is received, the server 130 is configured to determine whether to approve the request by reading the end/renewal date field. When time has not elapsed past the specified date, the server 130 is configured to transmit a message indicative that the request for medical equipment renewal is premature. The message may prompt the provider module 120 or the server 130 to provide follow up questions to determine if the current medical equipment is broken or lost and override the rejection. When the time has elapsed, the server 130 is configured to transmit an approval message, which enables the medical equipment to be released to the patient.

The medical equipment may include, for example, home based devices such as a physical therapy machine, a ventilator, an infusion pump, a dialysis machine, a heart rate monitor, a sleep monitor, etc. The medical equipment may also include disposable items such as line sets, catheters, needles, gauze, bandages, sample containers, etc. Further, in addition to medical equipment, the data structure template 800 may track home-based medical services such as physical therapy, wound cleaning/dressing, catheter replacement, etc.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A system for automatically verifying medical benefits information, the system comprising:
a memory device having a plurality of data structures, wherein the plurality of data structures include predetermined data fields that comprise at least medical benefit data fields and an insurance provider identifier data field; and
a server communicatively coupled to the memory device via a network, the server configured to:
receive a set of patient information associated with the patient from a healthcare provider computer over the network, wherein the set of patient information includes at least an insurance provider identifier;
create a patient account data structure using a data structure template by recording the insurance provider identifier received in the set of patient information into the insurance provider identifier data field, wherein the data structure template has the same data fields as the plurality of data structures in the memory device;
query the memory device for data structures with the same insurance provider identifier as the insurance provider identifier of the patient account;
after locating a data structure in the memory device with the same insurance provider identifier, automatically record benefits data in the medical benefits data fields of the data structure in the memory device into the medical benefits data fields of the patient account data structure; and
store the patient account data structure in a patient information database that is communicatively coupled via the network to the server.

2. The system of claim 1, wherein the set of patient information further comprises a first name, a last name, a date of birth, a member identification number, or a group identification number associated with the patient.

3. The system of claim 1, wherein the predetermined data fields of the plurality of data structures in the memory device and the data structure template comprise an insurance status, an insurance type, an insurance policy type, an insurance provider company name, a set of insurance company contact information, an insurance policy number, an insurance category number, an insurance group number, an insurance term date, a set of policyholder information, co-pay data, reimbursement data, deductible data, out-of-pocket data, a set of medical equipment guidelines, and a set of prescription authorizations.

4. The system of claim 1, wherein, when populating the medical benefit data fields of the patient account, the server when further configured to, after an unsuccessful match, automatically transmits a message to the healthcare provider computer, via the network, that prompts the healthcare provider to request medical benefit data from the patient.

5. The system of claim 1, wherein the insurance provider identifier is an insurance group identification number.

6. The system of claim 5, wherein the data structures in the memory device are organized by the insurance group identification number.

7. The system of claim 1, wherein the server is further configured to, after the patient account data structure is stored:
determine whether the insurance provider identifier stored within the patient account data structure corresponds to eligible insurance coverage provided by another healthcare provider computer or the same healthcare provider computer;
when the insurance provider identifier does not correspond to the eligible insurance coverage, transmit a hold message; and
when the insurance provider identifier corresponds to the eligible insurance coverage, transmit an indication of the eligible insurance coverage.

8. The system of claim 1, wherein the server is further configured to, after the patient account data structure is stored:
determine whether the benefits data stored within the patient account data structure corresponds to eligible insurance coverage provided by another healthcare provider computer or the same healthcare provider computer;
when the benefits data does not correspond to the eligible insurance coverage, transmit a hold message indicative that the patient does not possess necessary medical benefits; and
when the benefits data corresponds to the eligible insurance coverage, transmit an indication of the valid medical benefits.

9. The system of claim 8, wherein the server is further configured to add a verification note to the patient account data structure when the benefits data corresponds to the eligible insurance coverage.

10. The system of claim 9, wherein the verification note includes a date and/or time when the benefits data was verified.

11. A method for automatically verifying medical benefits information, the method comprising:
receiving, in a server, a set of patient information associated with the patient from a healthcare provider computer over a network, wherein the set of patient information includes at least an insurance provider identifier;
creating, via the server, a patient account data structure using a data structure template by recording the insurance provider identifier received in the set of patient information into an insurance provider identifier data field, wherein the data structure template includes predetermined data fields and the predetermined data fields comprise at least medical benefit data fields and an insurance provider identifier data field;
querying, via the server, the memory device for data structures with the same insurance provider identifier as the insurance provider identifier of the patient account;
after locating a data structure in a memory device with the same insurance provider identifier, automatically recording, via the server, the data in the medical benefits data fields of the data structure in the memory device into the medical benefits data fields of the patient account data structure; and
storing, via the server, the patient account data structure in a patient information database that is communicatively coupled to the server.

12. The method of claim 11, wherein the set of patient information further comprises a first name, a last name, a date of birth, a member identification number, or a group identification number associated with the patient.

13. The method of claim 11, wherein the predetermined data fields of the data structure template comprise an insurance status, an insurance type, an insurance policy type, an insurance provider company name, a set of insurance company contact information, an insurance policy number, an insurance category number, an insurance group number, an insurance term date, a set of policyholder information, co-pay data, reimbursement data, deductible data, out-of-pocket data, a set of medical equipment guidelines, and a set of prescription authorizations.

14. The method of claim 11, wherein, when populating the medical benefit data fields of the patient account, the method further comprises automatically transmitting, from the server via the network, a message to the healthcare provider computer that prompts the provider to request medical benefit data from the patient.

15. The method of claim 11, wherein the insurance provider identifier is an insurance group identification number.

16. The method of claim 15, wherein the data structures in the memory device are organized by the insurance group identification number.

17. The method of claim 11, further comprising:
determining, via the server, whether the insurance provider identifier stored within the patient account data structure corresponds to eligible insurance coverage provided by another healthcare provider computer or the same healthcare provider computer;
transmitting, via the server, a hold message when the insurance provider identifier does not correspond to the eligible insurance coverage; and
transmitting, via the server, an indication of the eligible insurance coverage when the insurance provider identifier corresponds to the eligible insurance coverage.

18. The method of claim 11, further comprising:
determining, via the server, whether the benefits data stored within the patient account data structure corresponds to eligible insurance coverage provided by another healthcare provider computer or the same healthcare provider computer;
transmitting, via the server, a hold message indicative that the patient does not possess necessary medical benefits when the benefits data does not correspond to the eligible insurance coverage; and
transmitting, via the server, an indication of the valid medical benefits when the benefits data corresponds to the eligible insurance coverage.

19. The method of claim 18, further comprising adding, via the server, a verification note to the patient account data structure when the benefits data corresponds to the eligible insurance coverage.

20. The method of claim 19, wherein the verification note includes a date and/or time when the benefits data was verified.
